(19) United States
(12) Patent Application Publication
(10) Pub. No.: US 2017/0367616 A1
(43) Pub. Date: Dec. 28, 2017

(54) System and Methods to Locate an Implanted Medical Device

(71) Applicant: Cardiac Pacemakers, Inc., St. Paul, MN (US)

(72) Inventors: Michael J. Kane, St. Paul, MN (US); Jean M. Bobgan, Maple Grove, MN (US); Keith R. Maile, New Brighton, MN (US)

(21) Appl. No.: 15/634,744

(22) Filed: Jun. 27, 2017

Related U.S. Application Data
(60) Provisional application No. 62/355,721, filed on Jun. 28, 2016.

(51) Int. Cl. A61B 5/06

(52) U.S. Cl. CPC A61B 5/062

(57) ABSTRACT

A system includes a sensor array having a plurality of sensors. Each sensor is coupled to circuitry configured to receive sensing signals from the plurality of sensors in the sensor array, where the sensing signals include a parameter indicating presence of an implantable medical device (IMD). Based on the received sensing signals, the circuitry is configured to generate display signals indicating location of the IMD.

### CROSS-REFERENCE TO RELATED APPLICATION

This application claims priority to Provisional Application No. 62/355,721, filed Jun. 28, 2016, which is herein incorporated by reference in its entirety.

### TECHNICAL FIELD

Certain embodiments of the present disclosure relate to implantable medical devices and systems, methods, and devices for locating implantable medical devices.

### BACKGROUND

Implantable medical devices (IMDs) may be configured to sense physiological parameters and/or provide therapy and may include one or more electrodes for performing aspects of these functions. IMDs may be implanted subcutaneously in a patient such as, for example, in a tissue pocket of the chest region. While implanted, an IMD may move from its initial position within the patient.

### SUMMARY

Certain embodiments of the present disclosure are accordingly directed to locating IMDs within a patient.

In Example 1, a system includes a sensor array having a plurality of sensors. Each sensor is coupled to circuitry configured to receive sensing signals from the plurality of sensors in the sensor array, where the sensing signals include a parameter indicating presence of an implantable medical device (IMD). Based on the received sensing signals, the circuitry is configured to generate display signals indicating location of the IMD.

In Example 2, the system of Example 1, wherein the circuitry is further configured to: subtract, from the received sensing signals, a reference signal to generate the display signals.

In Example 3, the system of either of Examples 1 or 2, wherein the circuitry is further configured to: multiplex the received sensing signals; compare a parameter of the multiplexed sensing signals to a threshold; and generate the display signals in response to the compared multiplexed sensing signals.

In Example 4, the system of Example 3, wherein the parameter of the multiplexed sensing signals is one of amplitude, frequency, and decay.

In Example 5, the system of any of Examples 1-4, wherein the sensor array comprises at least nine sensors.

In Example 6, the system of any of Examples 1-5, wherein the sensors comprise coils.

In Example 7, the system of any of Examples 1-6, wherein the sensors are coupled to a flexible circuit.

In Example 8, the system of any of Examples 1-7, further comprising: a display coupled to the circuitry and configured to receive the display signals.

In Example 9, the system of Example 8, wherein the display comprises a plurality of lights.

In Example 10, the system of any of Examples 1-9, further comprising: a housing, wherein the sensor array is positioned within the housing, and wherein the display is attached to the housing.

In Example 11, the system of any of Examples 1-9, further comprising: a flexible mesh, wherein the sensor array is coupled to the flexible mesh.

In Example 12, a method for use with a sensor array having a plurality of coils includes oscillating a first coil of the sensor array; detecting a parameter in a sensing signal of the first coil, the parameter being indicative of presence of an implantable medical device (IMD); performing oscillating and detecting steps using the other coils in the sensor array; and generating a display signal in response to the oscillating and detecting steps.

In Example 13, the method of Example 12, further comprising: subtracting a reference signal from each sensing signal to generate individual display signals.

In Example 14, the method of any of Examples 12-13, further comprising: displaying a location of the IMD on a display, in response to the individual display signals.

In Example 15, the method of any of Examples 12-14, further comprising: repeating the oscillating, detecting, performing, and displaying steps to update the display in real-time.

In Example 16, a system comprising a housing; a sensor array comprising a plurality of coil sensors, wherein the sensor array is positioned within the housing; a display attached to the housing; and circuitry, coupled to the sensor array and display/The circuitry is configured to: receive sensing signals from each coil sensor in the sensor array, wherein each sensing signal includes a parameter indicating presence of an implantable medical device (IMD), and based on the received sensing signals, generate display signals indicating location of the IMD.

In Example 17, the system of Example 16, wherein the display comprises a plurality of lights.

In Example 18, the system of any of Examples 16-17, wherein the display includes the same number of lights as the number of coil sensors.

In Example 19, the system of any of Examples 16-18, wherein the display is configured to receive the display signals, and wherein the lights are configured to indicate location of the IMD.

In Example 20, the system of any of Examples 16-19, further comprising: a transponder configured to communicate with an antenna of the IMD.

In Example 21, the system of any of Examples 16-20, further comprising a power source positioned with the housing.

In Example 22, the system of any of Examples 16-21, wherein the parameter is frequency, wherein the circuitry is further configured to: oscillate, at a first frequency, each coil sensor in the sensor array; detect a phase shift from the first frequency in each coil's sensing signal; and based on the detected phase shift, generate the display signals.

In Example 23, the system of Example 22, wherein the oscillating and detecting is carried out using a raster scan approach.

In Example 24, the system of any of Examples 16-23, wherein the circuitry is further configured to: subtract a reference phase shift from each detected phase shift to generate the display signals.

In Example 25, the system of any of Examples 16-24, wherein the housing is shaped and sized to be hand held.

In Example 26, the system of any of Examples 16-25, wherein the display comprising display means for indicating presence of the IMD.

In Example 27, a system comprising: a sensor array comprising a plurality of coil sensors; a flexible substrate coupled to the sensor array and including a plurality of light features; and circuitry coupled to the sensor array. The circuitry is configured to: receive sensing signals from the plurality of coil sensors, wherein the sensing signals include a parameter indicating presence of an implantable medical device (IMD). Based on the received sensing signals, the circuitry is configured to generate display signals indicating location of the IMD.

In Example 28, the system of Example 27, further comprising: at least one far field sensor coupled to the flexible substrate and configured to generate a far field sensing signal.

In Example 29, the system of any of Examples 27-28, wherein the circuitry is further configured to: subtract the far field sensing signal from the coil sensor's received sensing signals to generate the display signals.

In Example 30, the system of any of Examples 27-29, wherein the flexible substrate includes at least one cut out.

In Example 31, the system of any of Examples 27-30, wherein the light features are configured to indicate location of the IMD.

In Example 32, the system of any of Examples 27-31, wherein the parameter is frequency, wherein the circuitry is further configured to: oscillate, at a first frequency, each coil sensor in the sensor array; detect a phase shift from the first frequency in each coil's sensing signal; and based on the detected phase shift, generate the display signals.

In Example 33, the system of Example 32, wherein the oscillating and detecting is carried out using a raster scan approach.

In Example 34, the system of any of Examples 27-33, where in the flexible substrate comprises a mesh.

In Example 35, the system of any of Examples 27-34, wherein the flexible substrate is sized to be placed over a patient's chest.

While multiple embodiments are disclosed, still other embodiments of the present disclosure will become apparent to those skilled in the art from the following detailed description, which shows and describes illustrative embodiments of the disclosed subject matter. Accordingly, the drawings and detailed description are to be regarded as illustrative in nature and not restrictive.

### BRIEF DESCRIPTION OF THE DRAWINGS

FIG. 1 is a schematic illustration of a system including an implantable medical device (IMD), in accordance with certain embodiments of the present disclosure.

FIG. 2A is a top view of an implantable medical device (IMD), in accordance with certain embodiments of the present disclosure.

FIG. 2B is a lower perspective view of the IMD depicted in FIG. 2A, in accordance with certain embodiments of the present disclosure.

FIG. 3 is a side view of a portion of an IMD being gripped by a portion of a medical forceps, in accordance with certain embodiments of the present disclosure.

FIG. 4 is a schematic of an IMD detection system, in accordance with certain embodiments of the present disclosure.

FIG. 5 shows a flow chart of various methods, in accordance with certain embodiments of the present disclosure.

FIG. 6 is a schematic, front view of an IMD detection device, in accordance with certain embodiments of the present disclosure.

FIG. 7A is a schematic, back view of the IMD detection device of FIG. 6.

FIG. 7B is a schematic, back view of the IMD detection device of FIG. 6.

FIG. 8 is a schematic of an IMD detection device, in accordance with certain embodiments of the present disclosure.

While the invention is amenable to various modifications and alternative forms, specific embodiments have been shown by way of example in the drawings and are described in detail below. The intention, however, is not to limit the disclosed subject matter to the particular embodiments described. On the contrary, the disclosed subject matter is intended to cover all modifications, equivalents, and alternatives falling within the scope of the invention as defined by the appended claims.

Although the term “step” may be used herein to connote different elements illustratively employed, the term should not be interpreted as implying any requirement of, or particular order among or between, various steps disclosed herein. Similarly, although illustrative methods may be represented by one or more drawings (e.g., flow diagrams, communication flows, etc.), the drawings should not be interpreted as implying any requirement of, or particular order among or between, various steps disclosed herein. However, certain embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

### DETAILED DESCRIPTION

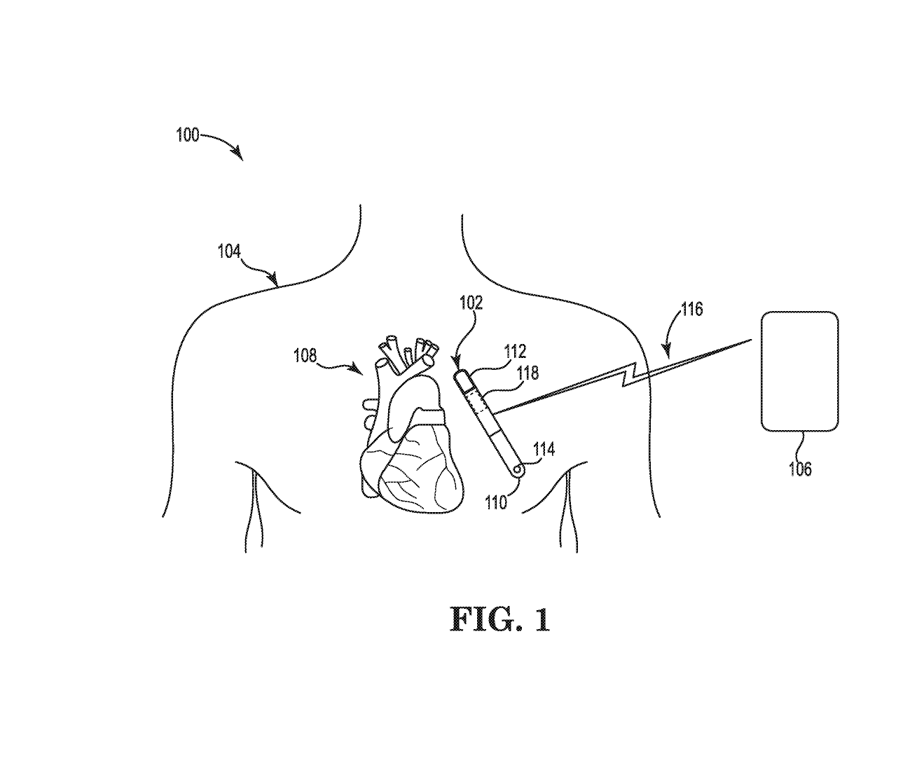

FIG. 1 is a schematic illustration of a system 100 including an implantable medical device (IMD) 102 implanted within a patient's body 104 and configured to communicate with a receiving device 106. The patient 104 may be a human, a dog, a pig, and/or any other animal having physiological parameters that can be recorded. In embodiments, the IMD 102 may be implanted subcutaneously within an implantation location or pocket in the patient's chest or abdomen and may be configured to monitor (e.g., sense and/or record) physiological parameters associated with the patient's heart 108. In embodiments, the IMD 102 may be an implantable cardiac monitor (ICM) (e.g., an implantable diagnostic monitor (IDM), an implantable loop recorder (ILR)) configured to record physiological parameters such as, for example, one or more cardiac activation signals, heart sounds, blood pressure measurements, oxygen saturations, and/or the like. That is, for example, the IMD 102 may be configured to measure electrical signals of the patient's heart, which may be used to ascertain heart rate, heart rhythms, and/or the like.

As shown, the IMD 102 may include a housing 110 having two electrodes 112 and 114 coupled thereto. According to embodiments, the IMD 102 may include any number of electrodes (and/or other types of sensors such as, e.g., thermometers, barometers, pressure sensors, optical sensors, motion sensors) in any number of various types of configurations, and the housing 110 may include any number of different shapes, sizes, and/or features. In embodiments, the IMD 102 may be configured to sense physiological parameters and record the physiological parameters. For example, the IMD 102 may be configured to activate (e.g., periodically, continuously, upon detection of an event, and/or the like), record a specified amount of data (e.g., physiological parameters) in a memory and communicate that recorded data to a receiving device 106 such as a programmer, controller, patient monitoring system, and/or the like.

In embodiments, the IMD 102 and the receiving device 106 may communicate through a wireless link. For example, the IMD 102 and the receiving device 106 may be coupled through a short-range radio link 116, such as Bluetooth, IEEE 802.11, a proprietary wireless protocol, and/or the like. In embodiments, for example, the radio link 116 utilize Bluetooth Low Energy radio (Bluetooth 4.1), or a similar protocol, and may utilize an operating frequency in the range of 2.40 to 2.48 GHz. The communications link may facilitate uni-directional and/or bi-directional communication between the IMD 102 and the receiving device 106. Data and/or control signals may be transmitted between the IMD 102 and the receiving device 106 to coordinate the functions of the IMD 102 and/or the receiving device 106. The term “communication link” may refer to an ability to communicate some type of information in at least one direction between at least two devices, and should not be understood to be limited to a direct, persistent, or otherwise limited communication channel. That is, according to embodiments, the communication link 116 may be a persistent communication link, an intermittent communication link, an ad-hoc communication link, and/or the like.

The communication link 116 may be facilitated, for example, by an antenna 118 disposed within, integrated with, and/or coupled to the IMD 102. The antenna 118 may include one or more antennas. The antenna 118 may be a bent monopole antenna, a patch antenna (e.g., a microstrip antenna, a planar inverted-F antenna (PIFA)), a slot antenna, a planar inverted-F antenna, a combination of these, a modification of one or more of these, and/or the like. According to embodiments, the antenna 118 may be disposed, at least in part, within the IMD 102, integrated with a portion of the housing of the IMD 102, be, or include, at least a portion of the housing of the IMD 102, and/or the like.

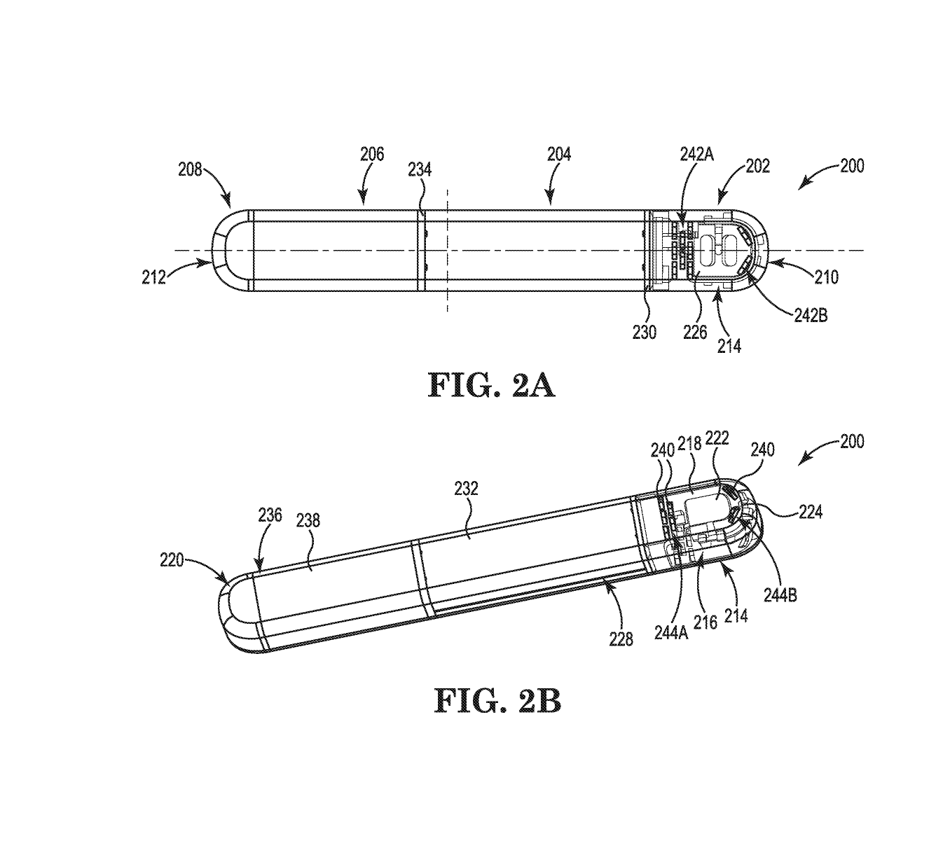

FIG. 2A is an upper view of an implantable medical device (IMD) 200, and FIG. 2B is a lower perspective view of the IMD 200, in accordance with embodiments of the disclosure. The IMD 200 may be, or may be similar to, the IMD 102 depicted in FIG. 1. As shown in FIG. 2A, the IMD 200 has a header 202, a core assembly 204, a battery assembly 206, and an electrode assembly 208. As shown in FIGS. 2A and 2B, the IMD 200 may have an elongated shape having a first end 210 and a second end 212.

The header 202 (which is illustrated as being transparent) is arranged at or near the first end 210 of the IMD 200. The header 202 includes a header housing 214 that encloses an interior region 216. As shown, an external surface 218 of the header housing 214 forms a portion of the outer surface 220 of the IMD 200. The header 202 may house various circuitry components within its interior region 216 (e.g., an electrode 222 and an antenna 224) positioned and supported by a scaffold assembly 226.

The core assembly 204 includes core circuitry enclosed within a core assembly housing 228. In embodiments, the core assembly 204 may be coupled to the header 202 via a feed-through assembly 230, which may be configured to provide a throughput for connections configured to connect the circuitry components of the header 202 to the core circuitry disposed within the core assembly 204. As shown, an external surface 232 of the core assembly housing 228 forms a portion of the outer surface 220 of the IMD 200.

The battery assembly 206 (which may include one or more batteries) is coupled to the core assembly 204. In embodiments, the battery assembly 206 may be coupled to the core assembly 204 via a feed-through assembly 234, which may be configured to provide a throughput for connections configured to connect the circuitry components of the core assembly 204 to the one or more batteries disposed within the battery assembly 206. The battery assembly 206 includes a battery assembly housing 236, and, as shown, an external surface 238 of the battery assembly housing 236 forms a portion of the outer surface 220 of the IMD 200. As shown, the electrode assembly 208 may form the second end 212 of the IMD 200, and may be coupled to the battery assembly 206.

As shown in FIGS. 2A and 2B, the outer surface 220 of the IMD 200 may incorporate or include additional features and structures such as, for example, protrusions 240 configured to inhibit migration of the IMD 200 within the patient's body, to facilitate gripping of the IMD 200 with a medical forceps, and/or the like. For example, the outer surface 220 of the IMD 200 may include any number of different types of protrusions 240 oriented in any number of different configurations, and having any number of different shapes. In the embodiments shown in FIGS. 2A and 2B, for example, protrusions 240 are disposed on the first surface 220 and the second surface 222.

As depicted in FIGS. 2A and 2B, a first set 242A and second set 242B of protrusions 240 are disposed on the external surface 218 of the header housing 214. Similarly, a third set 244A and a fourth set 244B of protrusions 240 are disposed on the external surface 218 of the header housing 214. According to embodiments, the first set 242A, second set 242B, third set 244A, and fourth set 244B of protrusions 240 each may include one or more protrusions 240 arranged in proximity to each other and oriented in any number of different configurations. Additionally, or alternatively, the IMD 200 may include any number of sets of protrusions (e.g., one set of protrusions, two sets of protrusions, three sets of protrusions, five sets of protrusions).

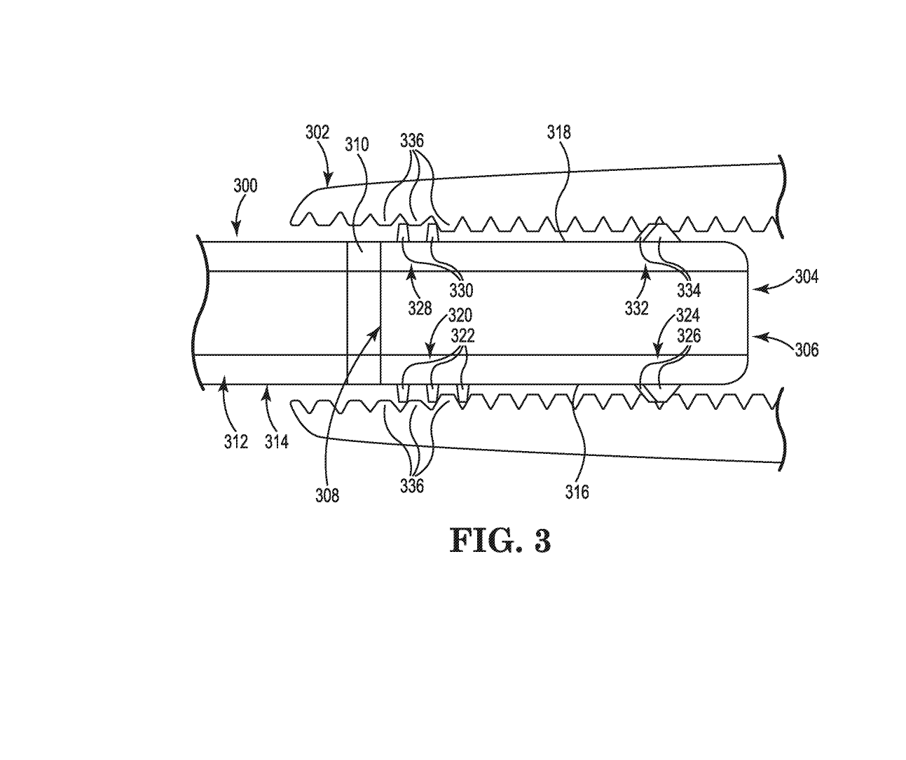

According to various embodiments, protrusions may be arranged in rows that are aligned to form multiple longitudinal columns of at least two protrusions. As shown in FIGS. 2A and 2B, the protrusions may be arranged in multiple rows, where each row is offset from an adjacent row. Any other arrangement of protrusions, rows of protrusions, columns of protrusions, and/or the like, may be implemented in accordance with embodiments of the disclosure. The protrusions may be formed according to any number of different shapes. For example, each protrusion may have a triangular cross sectional shape, having for example a widened base attached to the outer surface 220 of the IMD 200 and a narrowed apex, truncated side, or peak. In embodiments, each protrusion may have any number of different sizes, and in embodiments, the protrusions may be sized to fit within the grip of a medical forceps, as shown, for example, in FIG. 3.

FIG. 3 is a side view of a portion of an IMD 300 being gripped by a portion of a medical forceps 302, in accordance with embodiments of the disclosure. The IMD 300 may be, be identical to, or be similar to, the IMD 102 depicted in FIG. 1 and/or the IMD 200 depicted in FIGS. 2A and 2B. As shown, for example, in FIG. 3, the IMD 300 includes a header 304 having a first end 306 and a second end 308. The second end 308 of the header 304 is coupled, via a feed-through assembly 310 to a core assembly 312.

A first set 320 of protrusions 322 is disposed on the first surface 316 near the second end 308 of the header 304; a second set 324 of protrusions 326 is disposed on the first surface 316 near the first end 306 of the header; a third set 328 of protrusions 330 is disposed on the second surface 318 near the second end 308 of the header; and a fourth set 332 of protrusions 334 is disposed on the second surface 318 near the first end 306 of the header 304. As shown, the protrusions 322, 326, 330, and 334 are shaped and arranged to correspond to the shape and arrangement, respectively, of the teeth 336 of the medical forceps 302. For example, the width of each protrusion may be sized to fit within each of the spaces in the grips of a medical forceps; and the height of each protrusion may be sized to be received a distance into the grip of a medical forceps. In embodiments, the protrusions 322, 326, 330, and 334 may be sized with a complementary height, length, and/or width to the teeth 336 of a standard medical forceps, a custom medical forceps, and/or the like. In embodiments, the protrusions 322, 326, 330, and 334 may be configured in different sizes so that at least one of the protrusions 322, 326, 330, and 334 corresponds to one of several different styles, sizes, and/or shapes of forceps. The protrusions 322, 326, 330, and 334 facilitate gripping of the IMD 300 when the IMD 300 is being extracted from a patient.

As previously mentioned, IMDs may be implanted subcutaneously in a patient such as in a tissue pocket of the chest region. While implanted, an IMD may move from its initial position within the patient. Because IMDs are sized to be relatively small, movement of the IMD within the patient can make extraction difficult because a physician may not be able to accurately locate an IMD and therefore may not know where to make an incision to extract the IMD from the patient.

Typically, to extract an IMD, a physician will first palpate a patient's skin near an initial incision point where the IMD was implanted to see if the physician can feel the IMD. If the IMD has migrated from the initial incision point or deeper into a patient, palpation may not effectively locate the IMD. If palpation is not successful, the physician may try to locate the IMD by using an x-ray, which has drawbacks such as added expense, exposing the patient to radiation, and the challenge of using some reference point that shows up on both the patient and in the x-ray. Even when an IMD can be located using x-ray, it may be difficult to know how the IMD is oriented. Certain embodiments of the present disclosure are accordingly directed to assisting with locating IMDs implanted within patients.

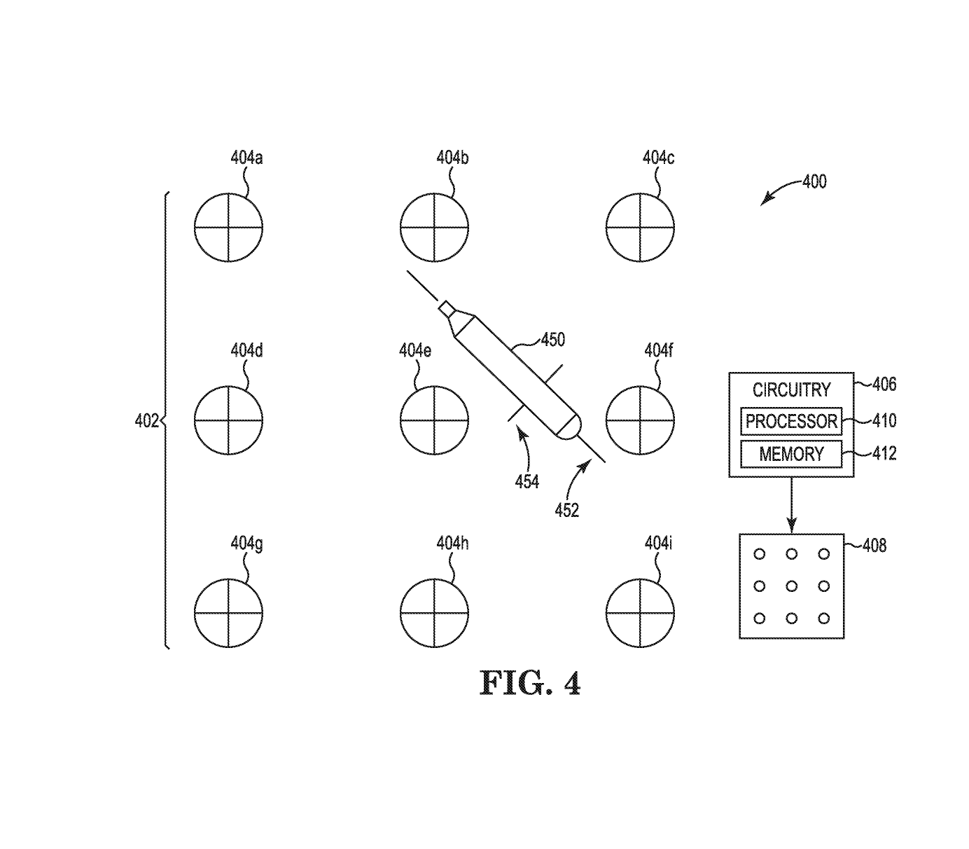

FIG. 4 shows an IMD locating system 400 and an IMD 450. The system 400 includes a sensor array 402 with each sensor 404a-i communicably coupled to circuitry 406, which is communicably coupled to a display 408. Although nine sensors are shown in the sensor array 402, it is contemplated that the sensor array 402 can include fewer (e.g., at least two sensors) or more sensors. Each of the sensors 404a-i is configured to sense a presence of the IMD 450. For example, each sensor 404a-i can output a sensing signal including a parameter (e.g., amplitude, frequency, decay) that indicates the presence of the IMD 450. The sensing signals can be used to determine a location of the IMD 450 within a patient. In embodiments, the sensors 404a-i in the sensor array 402 can be arranged to identify an orientation of the IMD 450. For example, it is useful to distinguish between, or identify, the long axis 452 and the short axis 454 of the IMD 450. The sensors 404a-i are communicably coupled to the circuitry 406 such that the sensor's sensing signals can be multiplexed (discussed in more detail below) and used to provide real-time feedback of an IMD's location with respect to the IMD locating system 400.

In some embodiments, the sensors are inductive coils configured to sense the presence of metallic objects within a certain proximity. In embodiments, a pulsing current is applied to each coil (e.g., oscillation), which induces a magnetic field. When the induced magnetic field interacts with a metallic object, the magnetic field induces electric currents (e.g., eddy currents) in the metallic object. The induced electric currents generate an opposite current in the coils, which induces a sensing signal with various parameters (e.g., amplitude, frequency, decay) that can indicate the presence of the metallic object. In some embodiments, the sensors are configured to sense high frequencies (e.g., radio frequencies). For example, the sensors can be radio-frequency antennas or coil-based sensors. Detecting higher frequencies may allow use of smaller sensors (e.g., diameter of coils), which can increase resolution as the sensors are spaced closer together. In embodiments, a combination of inductive sensors and radio-frequency sensors may be used.

The sensors 404a-i and the arrangement of the sensor array 402 can be designed with application-specific characteristics. For example, in the application of IMD detection using inductive coil sensors, the coils can have a specific number of windings, sizes (e.g., penny-size, dime-size), shapes, etc., that dictate the coils' level of sensitivity, which affects the coils' ability to sense metallic objects whether the object is the intended metallic object (i.e., IMD) or unintended metallic object (e.g., heart valves, staples, stents, operating table). Having multiple sensors allows for differential signaling such that the system 400 (and its circuitry 406) is capable of cancelling background inductances caused by objects like surgical tables. Moreover, having a sensor array 402 of relatively-small sensors 404a-i can reduce far-field sensitivity while maintaining spatial sensitivity. Although the sensors 404a-i in the sensor array 402 are shown as being vertically aligned with each other from row to row, the disclosure is not limited to such arrangements. For example, the sensors 404a-i can be offset from each other, be arranged in a circular, rectangular, and/or diamond pattern, etc. The sensors 404a-i can be positioned in a substantially planar, coplanar, perpendicular arrangement, etc. In certain embodiments, for IMD detection, the coils are designed with a sensitivity such that the IMD locating system detects metallic items at a depth ranging from 8 mm to 10 cm.

The circuitry shown and described in the figures can be implemented using firmware, integrated circuits, and/or computer-readable instructions/code for execution by a processor—with the various circuitry components able to interact with each other or be combined together. For example, the functions and described herein may be used to create computer-readable instructions/code for execution by a processor 410. Such instructions may be stored on a non-transitory computer-readable medium (e.g., memory 412) and transferred to the processor 410 for execution.

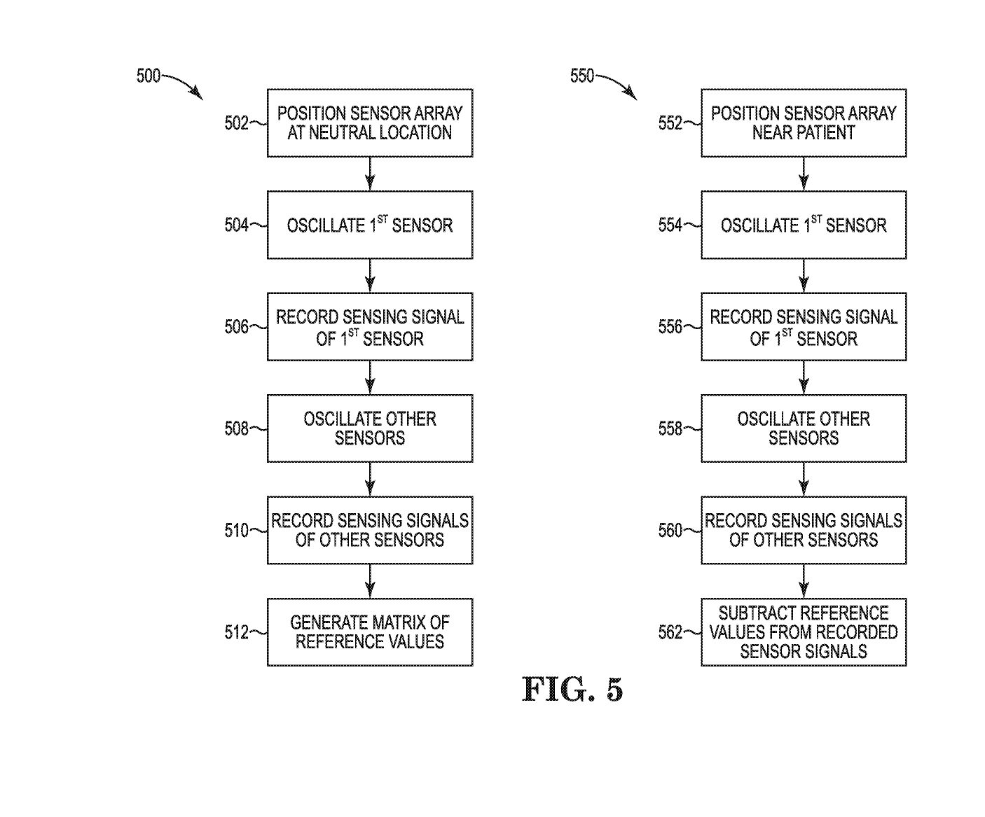

FIG. 5 shows a flow chart of various methods 500, 550 for use—individually or collectively—with the IMD locating system 400. The method 500 includes steps that assist with cancelling out interference or background noise by generating a reference value for each sensor. Step 502 involves placing the sensor array 402 in a neutral location. The neutral location can be a location where there are little to no metallic or magnetic objects within a certain distance (e.g., 1 meter) from the IMD locating system 400. In some embodiments, the neutral location can be a fixed distance from a surgical table. For example, to account for sensing signals caused by a metallic surgical table, the IMD locating system 400 could be placed at a location that is coplanar with the surgical table and at a distance equivalent to the depth of a patient's torso (e.g., 25 to 50 cm depending on patient). In some embodiments, the neutral location is a location with a known magnetic field.

Once in the neutral location, the IMD locating system 400 initiates a routine where a first sensor 404a is oscillated (step 504) and its responsive sensing signal is recorded (step 506). The recorded sensing signal can include the signal's parameters such as amplitude, frequency, decay, etc. The same oscillating and recording steps are carried out for the remaining sensors 404b-i in the sensor array (steps 508 and 510) using a raster scan approach. The recorded sensing signal parameters are used to create a matrix (step 512), which represents a reference value for each sensor 404a-i in the sensor array 402. The reference value represents a baseline amount of noise and/or interference sensed by each sensor 404a-i. The matrix of reference values can be used while the IMD locating system 400 is being used to sense and locate an IMD implanted within a patient.

The method 550 includes steps that repeatedly multiplex and compare sensing signals to sense for and display an IMD's location and/or orientation within a patient. Step 552 involves placing the sensor array 402 near a patient. Once near the patient, the first sensor 404a is oscillated (step 554) and its responsive sensing signal is recorded (step 556). The recorded sensing signal can include the signal's parameters such as amplitude, frequency, decay, etc. For example, a frequency of the recorded sensing signal may have been shifted from the oscillating frequency which indicates proximity of a metallic object. The same oscillating and recording steps are carried out for the remaining sensors 404b-i in the sensor array (steps 558 and 560) in a raster scan approach. The matrix of reference values is subtracted from the recoded sensing signal parameters to generate display signals (step 562). Steps 552-560 are then repeated at a desired frequency (e.g., 5 Hz) such that the display signals are repeatedly updated for real-time feedback of an IMD's location with respect to the device 400.

The display signals are used to generate a visual display (discussed in more detail below) to guide a user to an IMD's location within a patient. The display signals (and therefore display 408) can be continuously or continually updated as the sensor array 402 is moved across a patient's body. For example, as the sensor array 402 is moved closer to an implanted IMD, sensors closest to the IMD will generate sensing signals with parameters (e.g., amplitude, phase shift, decay) indicative of the sensors' proximity to the IMD when compared to other sensors positioned farther away from the IMD. In the embodiment shown in FIG. 4, sensors 404b, 404e, and 404f would generate sensing signals that, when compared to sensing signals of the other sensors, would indicate that the IMD 450 is closest to the sensors 404b, 404e, and 404f. As will be discussed in more detail below, the display 408 would then indicate, via visual or audio feedback, where the IMD is located to the user of the IMD locating system 400.

In certain embodiments, the display signals can be generated by comparing relative peaks and nulls of sensing signal parameters on paired combinations of sensors. In embodiments, the sensing signal parameters include amplitude, frequency, decay, etc. In embodiments, an average of certain sensing signal parameters is calculated and individual sensing signal parameters are compared to the average to determine whether the sensing signal is indicative of the presence of a metallic object. In embodiments, individual sensing signal parameters are compared to a predetermined or dynamically-set threshold to determine whether the sensing signal is indicative of the presence of a metallic object.

Although FIG. 5 outlines one method 500 for assisting with cancelling out interference and/or noise, other methods can be used in conjunction with the method 500, or independently, to mitigate interference and/or noise. In certain embodiments, the circuitry 406 can include one or more filters (e.g., bandpass, low pass, high pass, notch) designed to filter out unwanted signal content. For example, a notch filter could be implemented to attenuate certain signal content representative of objects known to interfere with detecting IMDs. In certain embodiments, the IMD 450 has a unique electro-magnetic signature that the IMD locating system 400 can be configured to detect. For example, certain filter parameters can be used to attenuate sensing signal content that does not match the IMD's electro-magnetic signature. In certain embodiments, the IMD locating system can be programmed to recognize certain patterns and/or be programmed to learn to recognize certain patterns over time. For example, when the IMD locating system 400 senses objects that do not match a shape and/or size of an IMD, the IMD locating system 400 could automatically cancel out and/or not display such sensed signals.

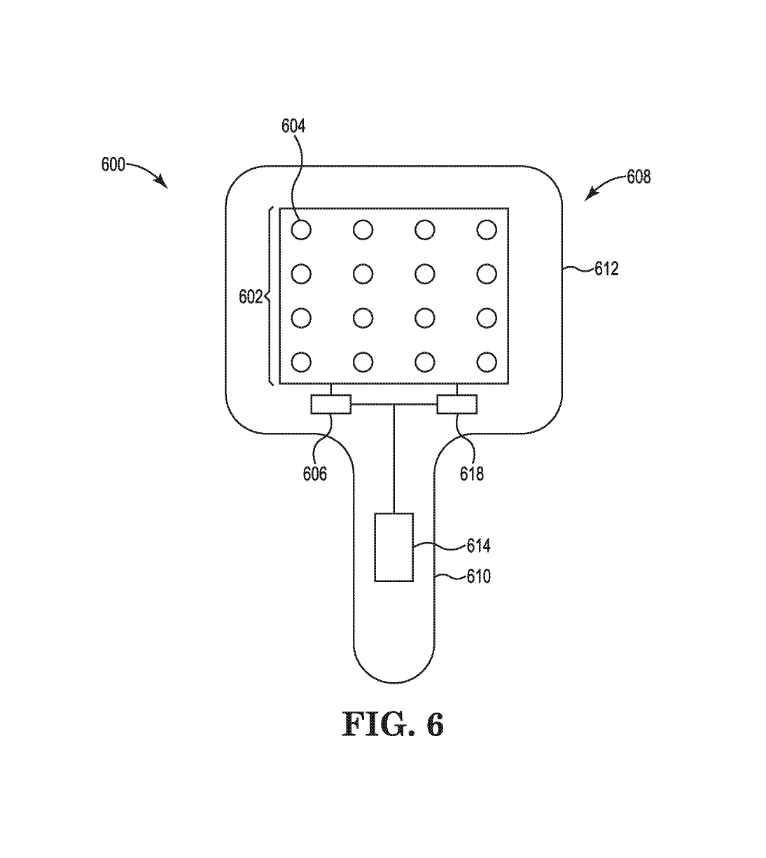

FIG. 6A shows a schematic of a hand-held IMD detection device 600, which can be used to scan over a patient's body and identify where an implanted IMD is located. The device 600 includes a sensor array 602 with each sensor 604 coupled to circuitry 606. The sensors 604 can be inductive coils, which may be coupled to a flex-circuit or substantially planar printed circuit board. The coils can include windings wound in a direction co-planar with the flex-circuit or printed circuit board such that the coils generate magnetic fields in a direction perpendicular to the flex-circuit or printed circuit board.

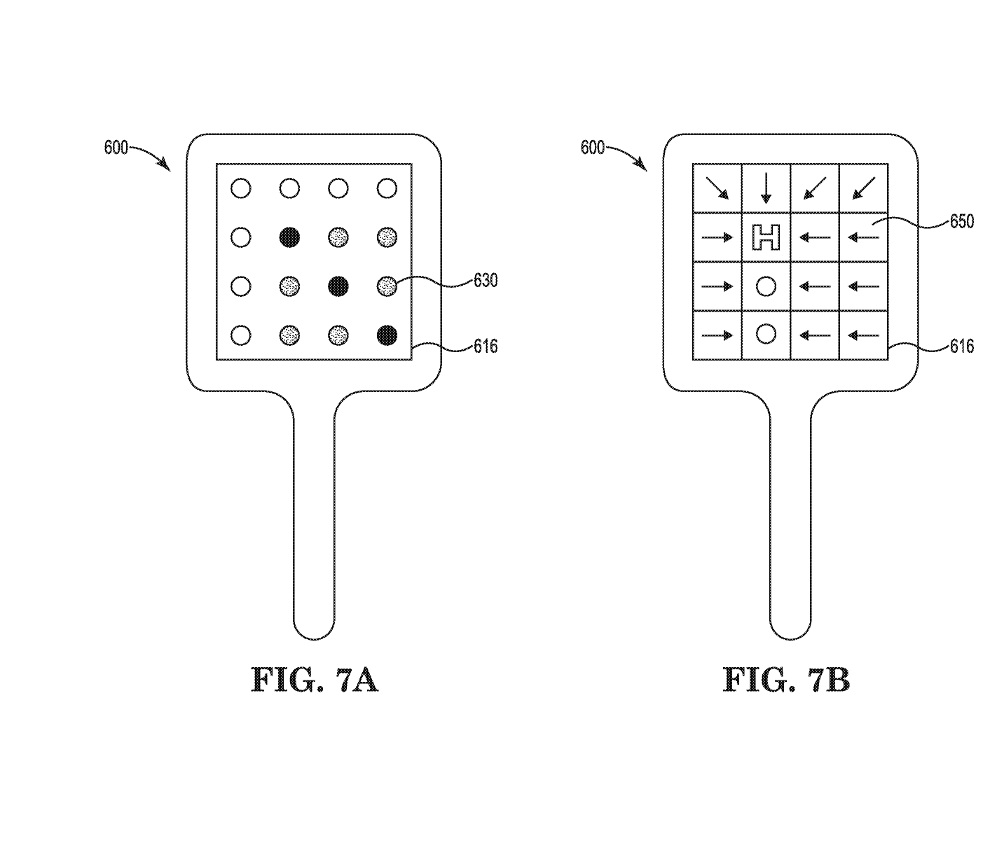

The sensor array 602 is positioned within a housing 608 of the device 600 which is sized and shaped to be a hand-held device. The housing 608 may also be shaped to match the shape and size of a human torso. It is appreciated that the housing 608 may be a variety shapes and the disclosure is not limited to the specific embodiments disclosed herein. The housing 608 includes a handle section 610 and a sensing section 612 in which the sensor array 602 is positioned. The device 600 also includes a power source 614 such as a terminal for electrically connecting batteries to the circuitry 606. The circuitry 606 and power source 614 are coupled to a display 616 (shown in FIGS. 7A-C) and a transponder 618, which is configured to communicate with an antenna of the IMD (for example antenna 118 of FIG. 1) by utilizing Bluetooth Low Energy radio (Bluetooth 4.1), or a similar protocol.

FIGS. 7A-B show schematics of the hand-held IMD detection device 600 and, in particular, various configurations of the display 616. FIG. 7A shows the display 616 having lights 630 (e.g., LEDs) where the device 600 includes as many lights 630 as there are sensors 604 (e.g., sixteen lights and sixteen sensors). In this embodiment, each light 630 is associated with and positioned in alignment with one sensor 604. In operation, as the device 600 (and therefore sensor array 602) is positioned over a patient, each sensor 604 is configured to output a sensing signal indicative of the presence of a metallic object. The sensing signals can be used by the circuitry 606 (as discussed above) to generate display signals that control aspects of the lights 630. For example, as a sensor 604 moves closer to a metallic object, the display signals can cause the lights to increase in brightness, change color, flash and/or increase frequency of flashing, turn on, etc. to indicate the presence of the metallic object.

The display 616 in FIG. 7A shows three lights (e.g., blackened lights) as having a high brightness, a number of surrounding lights (e.g., grayed lights) as having a lesser brightness, and the remaining lights (e.g., white lights) without any brightness. This display would indicate that an IMD is positioned most closely to the brightest three lights. This display may also indicate the IMD's orientation, where the IMD's long axis is aligned with a direction between a bottom-right side of the display to a top-left side of the display. In addition to indicating the long axis of the IMD, the device's transponder 618 can be used to communicate with an antenna of the IMD to indicate which side of the IMD the IMD's header 202 (see FIG. 2) is located. As previously discussed, the header 202 can include extraction features such as protrusions for assisting with extracting the IMD. As such, it would be useful for the display 616 to indicate which side the extraction features is located. Such indication could include increasing brightness of the light closest to the IMD's header or causing the closest light to blink or change colors. Other configurations are contemplated and are within the scope of the present disclosure. In some embodiments, the sensing signals are compared against a threshold such that only lights associated with sensors outputting sensing signals greater than the threshold become lit.

Using such a device and display can assist a physician with identifying the location of an implanted IMD. For example, upon identifying a location and orientation of an IMD, the physician could mark a patient's skin to identify a point where to make an incision to extract the IMD.

FIG. 7B shows the display 616 having a 4×4 grid where each grid tile 650 is associated with and positioned in alignment with one sensor 604. In operation, as the device 600 (and therefore sensor array 602) is positioned over a patient, each sensor 604 is configured to output a sensing signal indicative of the presence of a metallic object. The sensing signals can be used by the circuitry 606 (as discussed above) to generate display signals that control aspects of the tiles 650. For example, as a sensor 604 moves closer to a metallic object, the display signals can cause the tiles to perform similarly to the lights 616 shown in FIG. 7A (e.g., increase in brightness, change color, flash and/or increasing frequency of flashing, turn on) to indicate the presence of the metallic object. In other embodiments, each tile is designed to display numbers, letters, and/or shapes that indicate the presence of a metallic object. For example, as shown in FIG. 7B, certain tiles display an arrow indicating a location of an IMD with respect to the device 600. Using the transponder 618, the tile closest to the IMD could display a number, letter, and/or shape (e.g., the letter “H” is shown in FIG. 7B) to indicate a location of the IMD's header and therefore extraction features.

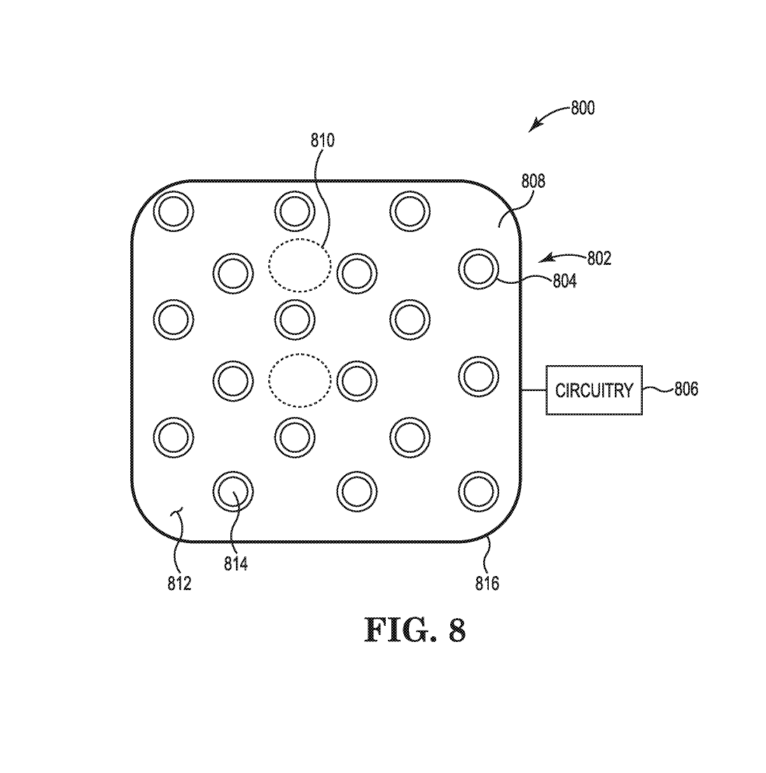

FIG. 8 shows a schematic of an IMD detection device 800, which can be placed over a patient's body and used to identify where an implanted IMD is located. The device 800 includes a sensor array 802 with each sensor 804 coupled to circuitry 806. The sensors 804 are coupled to a flexible substrate 808 (e.g., polyimide material) that is capable of conforming to the patient's body. The flexible substrate 808 may have cut-outs 810 that allow for access to a patient's skin for palpation, for adding a mark to identify location of the IMD, and/or for making an incision for extracting an IMD.

The device 800 can include a mesh 812 with light features 814 (e.g., LEDs) that assist with locating an IMD. The mesh 812 can be flexible and be encased in a molder silicone or laminated flexible circuit. For example, the mesh 812 can be an encapsulated woven mesh. The device 800 can also include at least one far field sensor 816 positioned around the sensor array 802 such that sensing signals generated by the far field sensor 816 can be used to cancel far field signals sensed by the sensors 804. For example, FIG. 8 shows the far field sensor 816 positioned along an entire perimeter of the flexible substrate 808.

In operation, upon placing the device 800 over a patient's chest, the light features 814 could indicate location of an implanted IMD by utilizing aspects of the light features (e.g., color, brightness, flashing and/or increasing frequency of flashing, turn on). For example, light features 814 positioned closest to the IMD could shine a certain color or at a certain brightness—compared to light features 814 positioned further away from the IMD—to indicate location of the IMD. The device 800 could be moved over the patient's chest to align one or more of the cut-outs 810 with the IMD so that a physician could identify the IMD's location by marking the patient's skin or even make an incision to extract the IMD while the device is positioned on the patient.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A system comprising:
a housing;
a sensor array comprising a plurality of coil sensors, wherein the sensor array is positioned within the housing;
a display attached to the housing; and
circuitry, coupled to the sensor array and display, and configured to:
receive sensing signals from each coil sensor in the sensor array, wherein each sensing signal includes a parameter indicating presence of an implantable medical device (IMD), and
based on the received sensing signals, generate display signals indicating location of the IMD.

2. The system of claim 1, wherein the display comprises a plurality of lights.

3. The system of claim 2, wherein the display includes the same number of lights as the number of coil sensors.

4. The system of claim 2, wherein the display is configured to receive the display signals, and wherein the lights are configured to indicate location of the IMD.

5. The system of claim 1, further comprising:
a transponder configured to communicate with an antenna of the IMD.

6. The system of claim 1, further comprising a power source positioned with the housing.

7. The system of claim 1, wherein the parameter is frequency, wherein the circuitry is further configured to:
oscillate, at a first frequency, each coil sensor in the sensor array;
detect a phase shift from the first frequency in each coil's sensing signal; and
based on the detected phase shift, generate the display signals.

8. The system of claim 7, wherein the oscillating and detecting is carried out using a raster scan approach.

9. The system of claim 7, wherein the circuitry is further configured to:
subtract a reference phase shift from each detected phase shift to generate the display signals.

10. The system of claim 1, wherein the housing is shaped and sized to be hand held.

11. The system of claim 1, wherein the display comprising display means for indicating presence of the IMD.

12. A system comprising:
a sensor array comprising a plurality of coil sensors;
a flexible substrate coupled to the sensor array and including a plurality of light features; and
circuitry coupled to the sensor array and configured to:
receive sensing signals from the plurality of coil sensors, wherein the sensing signals include a parameter indicating presence of an implantable medical device (IMD), and
based on the received sensing signals, generate display signals indicating location of the IMD.

13. The system of claim 12, further comprising:
at least one far field sensor coupled to the flexible substrate and configured to generate a far field sensing signal.

14. The system of claim 13, wherein the circuitry is further configured to:
subtract the far field sensing signal from the coil sensor's received sensing signals to generate the display signals.

15. The system of claim 12, wherein the flexible substrate includes at least one cut out.

16. The system of claim 12, wherein the light features are configured to indicate location of the IMD.

17. The system of claim 12, wherein the parameter is frequency, wherein the circuitry is further configured to:
oscillate, at a first frequency, each coil sensor in the sensor array;
detect a phase shift from the first frequency in each coil's sensing signal; and
based on the detected phase shift, generate the display signals.

18. The system of claim 17, wherein the oscillating and detecting is carried out using a raster scan approach.

19. The system of claim 12, where in the flexible substrate comprises a mesh.

20. The system of claim 12, wherein the flexible substrate is sized to be placed over a patient's chest.
